(19) United States
(12) Patent Application Publication
(10) Pub. No.: US 2017/0268155 A1
(43) Pub. Date: Sep. 21, 2017

(54) DEVICE FOR TREATING STAINS AND METHOD FOR TREATING STAINS USING SUCH A DEVICE

(71) Applicant: Henkel AG & Co. KGaA, Duesseldorf (DE)

(72) Inventors: Iwona Spill, Berlin (DE); Peter Schmiedel, Duesseldorf (DE); Michael Dreja, Neuss (DE); Walter Heberlein, Wien (AT)

(73) Assignee: Henkel AG & Co. KGaA, Duesseldorf (DE)

(21) Appl. No.: 15/509,679

(22) PCT Filed: Sep. 10, 2015
(86) PCT No.: PCT/EP2015/070696

§ 371 Date: Mar. 8, 2017

(30) Foreign Application Priority Data
Sep. 10, 2014 (DE) 10 2014 218 064.3

(51) Int. Cl. D06F 43/00; B08B 7/00; C11D 11/00; D06F 35/00

(52) U.S. Cl. CPC D06F 43/002; D06F 35/00; B08B 7/0057; C11D 11/007

(57) ABSTRACT

The disclosure relates to a device for treating stains, which can be carried by a person, comprising a reservoir for receiving a chemical formulation, where the reservoir has an outlet port for dispensing the chemical formulation and where the device comprises a UV radiation source which emits its radiation in a direction essentially away from the device. The invention further relates to a method for treating spots using such a device.

### CROSS-REFERENCE TO RELATED APPLICATION

This application is a U.S. National-Stage entry under 35 U.S.C. §371 based on International Application No. PCT/EP2015/070696, filed Sep. 10, 2015, which was published under PCT Article 21(2) and which claims priority to German Application No. 10 2014 218 064.3, filed Sep. 10, 2014, which are all hereby incorporated in their entirety by reference.

### TECHNICAL FIELD

The present disclosure relates to a device for treating stains and to a method for treating stains using a device of this kind.

### BACKGROUND

The disclosure addresses the removal of stains, in particular from textiles such as clothing, bed linen, table linen and the like. Locally restricted stains, for example from fruit, red wine, cosmetics, etc., often occur in situations in which it is not possible to immediately change the textile and put it in the wash. This may be the case for example when travelling or at work. It is desirable for it to be possible to treat stains of this type in a local and prompt manner and independently of a regular wash cycle.

Furthermore, in many cases, stubborn stains are also only inadequately eliminated in a normal wash cycle in the washing machine, in particular when low washing temperatures are selected. Since many textiles cannot be washed at high temperatures owing to the material composition thereof, in these cases, there needs to be alternative or additional treatment in order to remove these stains or at least make them fade.

U.S. Pat. No. 7,596,974 B2 discloses a device and a method for immediately removing stains, comprising a liquid reservoir for receiving a stain treatment liquid, and an absorbent material, the stain treatment liquid being applied to the stain and being at least partially removed by means of the absorbent material once the stain has been completely or partially dissolved. However, the results obtained by treating stains in this way are often inadequate. In particular, owing to its composition, a particular stain treatment liquid is effective preferably against a particular type of stain, but has little effect on other kinds of soiling.

The patent application DE 102013226835.1 describes a washing machine comprising a decoloration reservoir and a method for washing textiles in such a washing machine. The decoloration reservoir comprises an activation means, for example a UV radiation source, which is suitable for initiating a process for forming free radicals within the decoloration reservoir. Dyes in the washing liquid can be degraded by these free radicals.

### BRIEF SUMMARY

Methods of devices for treating stains are provided herein. In an embodiment, a device for treating stains is provided which can be carried around on its own by a person. The device includes a reservoir and a UV radiation source. The reservoir is provided for receiving a chemical formulation. The reservoir includes an outlet opening for dispensing the chemical formulation. The UV radiation source emits radiation substantially in a direction away from the device.

In another embodiment, a method for treating stains is provided. The method uses a device that includes a reservoir and a UV radiation source. The reservoir receives a chemical formulation and includes an outlet opening for dispensing the chemical formulation. The UV radiation source emits radiation substantially in a direction away from the device. The method includes applying the chemical formulation from the reservoir to a stain to be treated. The stain that is provided with the chemical formulation is irradiated with UV radiation from the UV radiation source. Optionally, the treated stain is washed.

In another embodiment, a device for treating stains is provided. The device is pen-shaped with at least two parts that are connectable by a connecting mechanism. The device includes a reservoir and a UV radiation source. The reservoir is arranged in the first part for receiving a chemical formulation. The reservoir includes an outlet opening for dispensing the chemical formulation, and the device is compressible at least in the region of the reservoir. The UV radiation source is arranged in the second part of the device. The UV radiation source emits radiation substantially in a direction away from the device.

### BRIEF DESCRIPTION OF THE DRAWINGS

The present disclosure will hereinafter be described in conjunction with the following drawing figures, wherein like numerals denote like elements, and wherein:

FIG. 1 is a perspective view of an embodiment of the device according to the invention; and

FIG. 2 is a schematic view of a longitudinal section through the device from FIG. 1.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object as contemplated herein is that of providing efficient and easy-to-use measures which can be used to effectively treat stains of various origins in textiles even independently of a regular wash cycle.

This object is achieved by a device as described herein and by a method as described herein.

Advantageous embodiments and developments as contemplated herein are found in the dependent claims.

The subject matter as contemplated herein relates to a device for treating stains which can be carried around on its own by a person and comprises a reservoir for receiving a chemical formulation, the reservoir comprising an outlet opening for dispensing the chemical formulation, and the device comprising a UV radiation source which emits its radiation substantially in a direction away from the device.

In other words, the device as contemplated herein is characterized in particular in that it can be carried by a person and can therefore be used in everyday situations independently of a regular wash cycle. As contemplated herein, the wording “can be carried around [. . . ] on its own” is in this case to be understood to mean that the outer dimensions and the weight of the device are such that said device can be carried by a single person without excessively expending energy, for example in a handbag, in a rucksack or in a pants pocket. The device is thus extremely mobile and easy to carry when at work or when travelling, for example.

It is known that dyes can be decomposed by free radicals. Free radicals have at least one unpaired electron and for this reason are extremely reactive and therefore have a short life (generally <1 sec). They are able to react with dyes and to thus decompose said dyes. By way of example, the decomposition of the dye Acid Orange 7 is mentioned, which is decomposed by interaction with free radicals into colorless aromatic by-products, which are themselves converted into aliphatic acids by oxidation.

Free radicals are produced for example by UV irradiation of chemical substances which contain hydrogen peroxide (H₂O₂) or titanium dioxide (TiO₂). The hydrogen peroxide or titanium dioxide is activated by the UV radiation emitted by the radiation source and highly reactive hydroxyl radicals (OH radicals), which are capable of decomposing dyes, are produced as short-life products of this reaction. In addition to hydrogen peroxide and titanium dioxide, many other chemical substances which can be activated by UV radiation to form free radicals are suitable. The concentration of the chemical substances in the formulation is in a range of between about 0.1 and about 100%, preferably between about 5 and about 30%. The formulation may comprise a solvent.

The device for treating stains as contemplated herein makes use of this interactive behavior between free radicals and dyes. For this purpose, said device comprises the reservoir for receiving a chemical formulation, for example hydrogen peroxide or a mixture containing hydrogen peroxide, from which reservoir the chemical formulation can be applied to the stain to be treated via the outlet opening provided in the reservoir. Using the UV radiation source arranged on the device, the chemical formulation applied to the soiling can be irradiated, resulting in free radicals being formed. The formed free radicals attack the dyes in the soiling to be treated and decompose said dyes so that the stain disappears or at least fades.

The outstanding action of the free radicals on a wide range of dyes has proven to be particularly advantageous. The device as contemplated herein can therefore be used in an extremely versatile manner on various types of stains, and this distinguishes the device from other known stain treatment formulations.

According to an embodiment as contemplated herein, the length of the device is at most 20 cm, the width of the device is at most 10 cm and the height of the device is at most 6 cm. As a result of the dimensions being limited to these values, the device can easily be carried in conventional bags and rucksacks. The shape of the device may vary with different dimensions up to these maximums. Therefore, said device may instead be elongate or cuboidal, for example. It can, however, also be round or ellipsoidal.

The subject matter of the present disclosure proposes that the device is shaped like a pen having a first and a second end, the outlet opening in the reservoir being arranged in the region of the first end and the UV radiation source being arranged in the region of the second end of the device. As a result of this type of pen-like design, the device is easy to handle both when it is in use and when it is being transported.

In this regard, the length of a pen-shaped device of this kind, determined by the first and the second end, is preferably from about 5 to about 20 cm, particularly preferably from about 10 to about 15 cm. The average diameter of a device of this kind is preferably between about 0.5 and about 4 cm. In this case, the device can have a constant diameter over the entire length thereof, and can thus be cylindrical; however, said device can also have a conical or ellipsoidal outer shape or hybrid shapes. Different portions may also have different shapes.

According to an embodiment as contemplated herein, the device is designed having at least two parts, the reservoir being arranged in a first part of the device and the UV radiation source being arranged in a second part of the device, and it being possible to connect together the first part and the second part of the device by connecting mechanism. The first and the second part of the device are preferably screwable or lockable together.

When the device is designed having two parts or multiple parts, the reservoir can completely fill one part of the device and be identical to this part. However, the reservoir can also be designed as a sealed space within the corresponding part of the device.

The UV radiation source provided as contemplated herein may be for example at least one UV light-emitting diode, an arrangement of UV light-emitting diodes preferably having different wavelengths, or a UV quartz lamp. However, other UV radiation sources such as gas discharge lamps, fluorescent lamps, sunlight or lasers are also conceivable. In any case, as contemplated herein, the UV radiation source emits its radiation substantially in a direction away from the device. It is to be understood from this that at most a low amount of radiation is emitted back onto or into the device.

The preferred wavelength range of the emitted UV radiation is between 100 and 400 nm, particularly preferably between 250 and 400 nm. A wavelength of 254 nm, for example, is suitable for forming OH radicals from H₂O₂. Radicals from other compounds can optionally be formed at different wavelengths. As contemplated herein, the UV radiation source can emit radiation having a wide spectrum of wavelengths, but it can also use a plurality of light-emitting diodes for example that each emit radiation of a different wavelength.

As contemplated herein, the reservoir has a filling volume of from about 1 to about 50 ml. In this case, the reservoir can in principle and preferably be refillable. For this purpose, the reservoir can comprise, either in the region of the outlet opening or at a distance therefrom, a suitable opening through which the reservoir can be filled and refilled with the chemical formulation. However, the subject matter described herein also covers those devices which are filled with a corresponding chemical formulation just once during production and are disposed of once the reservoir is completely empty. Finally, particularly when the device is designed having at least two parts, it can also be provided that only the part of the device that contains the reservoir containing the chemical formulation is designed as a replaceable, disposable part, and therefore the part containing the UV radiation source can be repeatedly coupled to new single-use reservoir parts.

According to an embodiment as contemplated herein, the device is designed so as to be compressible in the region of the reservoir. This can be brought about by selecting a suitable material and results in the contents of the reservoir being dispensed in doses by a corresponding pressure being applied to the device.

In the region of the outlet opening, in particular around said opening, the reservoir can have a brush-like structure which makes it possible and easier to directly and gently rub the released formulation into the textile to be treated.

The device can comprise at least one protective cap for covering the UV radiation source and/or the outlet opening. In the region of the UV radiation source, this protective cap may be a transparent cover which is transparent to UV radiation, is not taken off even during operation and is used to protect the radiation source. The outlet opening can be covered by a protective cap which can be taken off so as to release the chemical formulation. The protective cap preferably comprises a seal for covering the outlet opening in order to prevent the chemical formulation from accidentally coming out of the device.

The protective cap may for example be provided with a thread or a locking means. The protective cap may also have a special childproof means in order to prevent children from accidentally coming into contact with the chemical formulation. In principle, when the protective cap is open, it can be taken off the device completely or it can remain on the device by means of a connecting piece or the like.

The at least one protective cap may be flat at its end which is at a distance from the device such that, when the protective cap is closed, the entire device can be put down on said cap. This improves the handleability of the device.

In order to operate the UV radiation source, as contemplated herein, the device comprises an internal battery holder and/or a terminal for producing a connection to an external voltage source. If the device is being used on the move, it can therefore be used in a flexible manner and independently of available voltage sources by being operated by means of batteries and/or rechargeable battery packs. If the device is being used at home, for example for pre-treating or post-treating stubborn stains, the device can also be connected to an external voltage source by means of an appropriate connection. For this purpose, the device may comprise an appropriate terminal.

The UV radiation source is preferably switched on and off by means of a switch which is arranged on the outside of the device.

According to embodiments, the subject matter as contemplated herein also relates to a method for treating stains using the device as described herein. The method includes the steps of:

applying the chemical formulation from the reservoir to a stain to be treated;

irradiating the stain provided with the chemical formulation in this way with UV radiation; and

optionally washing the treated stain out.

The last method step is used in particular when the method as contemplated herein is used to pre-treat stubborn stains and when a regular wash cycle is to take place following successful pre-treatment. However, using the method according to the invention, it is of course also possible to subsequently re-rinse treated stains with a small amount of water in order to remove any possible remaining residues of the chemical formulation.

According to embodiments as contemplated herein, the UV irradiation takes place for a period of from about 1 to about 60 minutes, preferably for a period of from about 5 to about 30 minutes. In this case, the extent to which the stain is removed generally depends on the duration of irradiation, and therefore the irradiation duration can be adapted to the intensity of the stain.

As contemplated herein, it is also possible to adapt the amount of chemical formulation used to the properties of the stain to be removed.

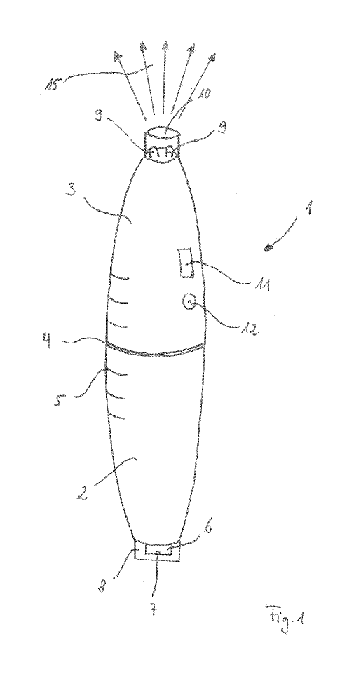

FIG. 1 shows an embodiment of a device as contemplated herein, which is denoted as a whole by reference numeral 1 and is used to treat stains. The device is designed having two parts and comprises a lower first part 2 and an upper second part 3. The part 2 and the part 3 of the device 1 are screwed together and, for this purpose, comprise corresponding threads on the end thereof which faces the other part. When screwed together, the two parts 2 and 3 abut one another in a flush manner in the region of the abutment edge 4.

The device 1 is about 14 cm long and is approximately spheroidal. The diameter of the device 1 is approximately 3.5 cm in the region of the abutment edge 4.

In order to improve the handleability of the device 1, said device comprises ribbing which is denoted by reference numeral 5, is intended to prevent the device 1 from accidentally slipping out of a user's hand and gives the device a certain degree of grip. In this case, the structure 5 can be around just some portions of the device, as shown in FIG. 1, or can even completely surround the device.

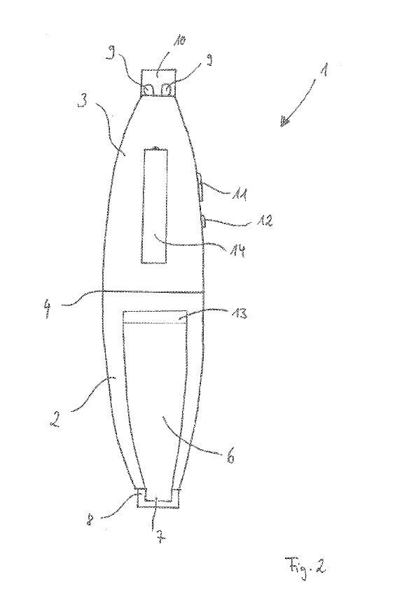

A reservoir 6 is arranged inside the lower part 2 of the device 1, only the lowermost portion of said reservoir, which portion protrudes out of the device 1, being visible in FIG. 1. The entire reservoir 6 can be seen in the sectional view in FIG. 2. The reservoir 6 is used to receive a chemical formulation, hydrogen peroxide in this case, and has a filling volume of about 20 ml.

The reservoir 6 comprises an outlet opening 7 from which the chemical formulation can be dispensed. In order to protect the outlet opening 7, a removable protective cap 8 is arranged thereon and comprises a seal (not shown). The protective cap 8 can be screwed to the device 1 and seals said device such that the chemical formulation is prevented from accidently coming out of the device.

The protective cap 8 is flat at the end thereof which faces away from the reservoir 6 so that, when the protective cap 8 is placed thereon, the entire device 1 can be put down on said cap.

Two UV light-emitting diodes 9 are arranged on the upper end of the upper part 3 of the device 1 and emit UV radiation in a wavelength range of from 240 to 380 nm. The UV light-emitting diodes 9 emit the UV radiation substantially in a direction away from the device 1, which direction is indicated by the group of arrows 15 in FIG. 1.

The light-emitting diodes 9 are supplied with the required operating voltage by means of a battery 14 which can be seen in FIG. 2 and is arranged inside the upper part 3 of the device. For the sake of clarity, the connections between the UV light-emitting diodes 9 and the battery 14 have not been included in the figure. The device 1 also comprises a connection means 12 for the connection to an external voltage source. The UV light-emitting diodes 9 can be switched on and off by means of a switch 11 arranged on the outside of the device 1. In order to cover the UV light-emitting diodes 9, the device 1 comprises a further protective cap 10, which is made of a material which is transparent to UV. The protective cap 10 is thus used to protect the light emitting-diodes 9 and stays in the described position even when the device 1 is being used. In the exact same manner as the protective cap 8 described above, the protective cap 10 is also flat at the end thereof which faces away from the upper part 3 such that the entire device 1 can be put down on said cap.

FIG. 2 shows that the reservoir 6 has an unscrewable closure means 13 on the end thereof which faces away from the outlet opening 7. In order to fill and refill the reservoir 6 with the chemical formulation, the first part 2 and the second part 3 of the device 1 are first unscrewed from one another. Then, the closure means 13 of the reservoir 6 is unscrewed and the reservoir 6 is filled with the formulation, which is hydrogen peroxide in this case, from a supply container. The closure means 13 is then closed again and the two parts 2 and 3 of the device 1 are screwed together.

In order to apply the chemical formulation to a stain to be treated, the protective cap 8 is taken off and a desired amount of the chemical formulation is removed from the reservoir via the outlet opening 7 by gently squeezing the device 1 in the region of the reservoir 6, and applied to the soiling. For this purpose, the lower part 2 of the device 1 and the reservoir 6 consist of a slightly flexible plastics material.

As soon as the desired amount of chemical formulation has been applied to the stain to be treated, the protective cap 8 is replaced and the device 1 is rotated by 180°. The UV light-emitting diodes 9 are then switched on by means of the switch 11 and the stain impregnated with the chemical formulation is irradiated with UV radiation. In this case, the duration of irradiation depends on how intense and stubborn the stain to be removed is. The hydrogen peroxide is activated by means of the irradiation with UV radiation and highly reactive hydroxyl radicals (OH radicals), which are capable of decomposing the dyes bound in the soiling, are produced as short-life products of this reaction. Once the stain has been irradiated for a period of typically from about 5 to about 30 minutes, it can be assumed that the stain has been removed or has faded to a substantial extent. During this irradiation period, the device 1 can be put down on the protective cap 10 in the region of the stain, and therefore, particularly when the irradiation period is long, the device 1 does not have to be held by hand.

Once the stain has been successfully treated, the treated region of the textile can be re-rinsed with clear water for example, in order to remove any possible residues of the chemical formulation. If the above-described method is used to pre-treat a stain, the textile can then be put into the normal wash.

The device 1 and the method using said device are thus efficient and easy-to-use measures which can be used to treat various stains in textiles even independently of a regular wash cycle. In particular, the device as contemplated herein, owing to its measurements and low weight, can be carried anywhere at all times.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A device for treating stains which can be carried around on its own by a person and comprises:
a reservoir for receiving a chemical formulation, the reservoir comprising an outlet opening for dispensing the chemical formulation,
wherein the device comprises a UV radiation source which emits radiation substantially in a direction away from the device.

2. The device according to claim 1, wherein the length of the device is at most about 20 cm, the width of the device is at most about 10 cm and the height of the device is at most about 6 cm.

3. The device according to claim 1, wherein the device is pen-shaped having a first and a second end, wherein the UV radiation source is arranged in a region of the first end and the outlet opening (7) in the reservoir is arranged in a region of the second end of the device.

4. The device according to claim 1, wherein the device has at least two parts, wherein the reservoir is arranged in a first part of the device and the UV radiation source is arranged in a second part of the device, and wherein the first part and the second part of the device are connectable by connecting mechanism.

5. The device according to claim 4, wherein the first part and the second part are screwable or lockable together.

6. The device according to claim 1, wherein the UV radiation source is chosen from a UV light-emitting diode and/or a quartz lamp.

7. The device according to claim 1, wherein the UV radiation source emits UV radiation in a wavelength range of from about 100 to about 400 nm.

8. The device according to claim 1, wherein the reservoir has a filling volume of from about 1 to about 50 ml.

9. The device according to claim 1, wherein the device is compressible at least in the region of the reservoir.

10. The device according to claim 1, further comprising at least one protective cap for covering the UV radiation source and/or the outlet opening.

11. The device according to claim 10, wherein the device comprises the protective cap for covering the outlet opening, and wherein the protective cap for covering the outlet opening has a seal.

12. The device according to claim 1, further comprising an internal battery holder and/or a terminal for producing a connection to an external voltage source.

13. The device according to claim 1, wherein a switch is provided for switching the UV radiation source on and off.

14. A method for treating stains using a device comprising a reservoir for receiving a chemical formulation, the reservoir comprising an outlet opening for dispensing the chemical formulation, wherein the device comprises a UV radiation source which emits radiation substantially in a direction away from the device, and wherein the method comprises the steps of:
applying the chemical formulation from the reservoir to a stain to be treated;
irradiating the stain provided with the chemical formulation in this way with UV radiation from the UV radiation source; and
optionally, washing the treated stain.

15. The method according to claim 14, wherein the stain is irradiated with the UV radiation for a period of from about 1 to about 60 minutes.

16. The device according to claim 1, wherein the UV radiation source emits UV radiation in a wavelength range of from about 250 to about 400 nm.

17. The method according to claim 15, wherein the stain is irradiated with the UV radiation for a period of from about 5 to about 30 minutes.

18. The method according to claim 15, wherein the device has at least two parts, wherein the reservoir is arranged in a first part of the device and the UV radiation source is arranged in a second part of the device, wherein the first part and the second part of the device are connectable by connecting mechanism, and wherein the chemical formulation is applied from the reservoir in the first part of the device.

19. The method according to claim 15, wherein the device is compressible at least in the region of the reservoir, and wherein applying the chemical formulation from the reservoir comprises compressing the reservoir.

20. A device for treating stains, wherein the device is pen-shaped with at least two parts connectable by a connecting mechanism and wherein the device comprises:
a reservoir arranged in the first part for receiving a chemical formulation, the reservoir comprising an outlet opening for dispensing the chemical formulation and wherein the device is compressible at least in the region of the reservoir, and
a UV radiation source arranged in the second part of the device, wherein the UV radiation source emits radiation substantially in a direction away from the device.
